(19) United States
(12) Patent Application Publication
(10) Pub. No.: US 2025/0171486 A1
(43) Pub. Date: May 29, 2025

(54) N4-ISOBUTYRYLOXYCYTIDINE ANALOG SYNTHESIS AND COMPOSITION FOR TREATING VIRAL INFECTION COMPRISING ANTI-VIRAL USE THEREOF

(71) Applicant: Dong-A University Research Foundation for Industry-Academy Cooperation, Busan (KR)

(72) Inventors: Jong Hyun CHO, Gimhae-si (KR); Yeon Jin AN, Ulsan (KR); Se Myeong CHOI, Busan (KR); Eun Rang CHOI, Busan (KR); Ye Eun NAM, Busan (KR); Eun Woo SEO, Changwon-si (KR)

(73) Assignee: Dong-A University Research Foundation for Industry-Academy Cooperation, Busan (KR)

(21) Appl. No.: 18/843,517

(22) PCT Filed: Sep. 13, 2022
(86) PCT No.: PCT/KR2022/013638

§ 371 Date: Sep. 3, 2024

(30) Foreign Application Priority Data
Mar. 4, 2022 (KR) 10-2022-0028064

(51) Int. Cl. C07H 19/067; A61K 31/7068; A61P 31/14; A61P 31/16

(52) U.S. Cl. CPC C07H 19/067; A61K 31/7068; A61P 31/14; A61P 31/16

(57) ABSTRACT

The present disclosure relates to N⁴-isobutyryloxycytidine analog synthesis and to the anti-viral use thereof against dengue virus, influenza virus, and SARS-COV-2 virus.

### TECHNICAL FIELD

The present disclosure relates to the synthesis of N⁴-isobutyryloxycytidine analogs and their antiviral uses.

### BACKGROUND ART

β-D-N⁴-hydroxycytidine (NHC) is a synthetic nucleoside that inhibits the polymerase of various RNA viruses, which acts as an inhibitor of high-risk RNA virus polymerase, but causes cytotoxicity and mutation of viral genes so that it has limitations in development as a novel antiviral drug.

Recently, NHC precursors have been approved by the U.S. Food and Drug Administration (FDA) as inhibitors of the polymerase of SARS-COV-2, but they still have problems with cytotoxicity and viral genetic mutations. Therefore, the derivation of new precursors that can reduce the cytotoxicity and mutations of NHCs is required.

### PRIOR ART LITERATURE

### Patent Document

Korea Patent No. 10-1228503.

### DISCLOSURE

### Technical Problem

Accordingly, the present inventors synthesized a novel N⁴-isobutyryloxycytidine, a prodrug mimicking the base portion of the nucleic acid of N⁴-hydroxycytidine (NHC), and confirmed its antiviral effect, thereby completing the present disclosure.

Therefore, the purpose of the present disclosure is to provide a synthesis of an N⁴-isobutyryloxycytidine analogs and their antiviral uses.

### Technical Solution

To achieve the above purpose, the present disclosure provides a compound represented by the following chemical formula 1 or a pharmaceutical salt thereof:

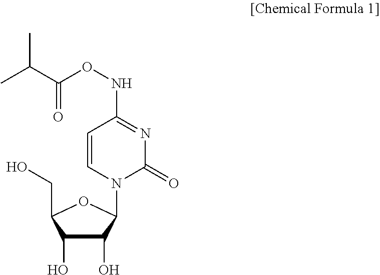

Further, the present disclosure provides an antiviral composition including the compound according to the present disclosure or a pharmaceutical salt thereof as an active ingredient.

### Advantageous Effects

The N⁴-isobutyryloxycytidine of the present disclosure has improved physiological activity compared to the parent NHC, exhibits higher cytotoxicity and antiviral efficacy against viruses, and thus can be used as an effective antiviral agent.

### Best Mode of the Invention

The present disclosure is a result of a project supported by Busan Metropolitan City in addition to one research project report recorded in the bibliographic information, and the research project information is as follows.
Project No.: 2021-0481
Ministry Name: Busan Metropolitan City
Project Management Agency Name: Busan Metropolitan City
Research Name: BB21 Plus Project
Research Project Name: Virus-Induced Geriatric Disease Treatment Specialist Training Project (4th Year)
Project Executing Agency: Dong-A University
Research Period: Jun. 1, 2021 to May 31, 2022

Hereinafter, embodiments according to the present disclosure are described in detail with reference to the accompanying drawings. In the following description, detailed descriptions of well-known technologies to those skilled in the art may be excluded. Further, in describing the present disclosure, if it is determined that a detailed description of related known function or configuration may unnecessarily obscure the subject matter of the present disclosure, the detailed description may be excluded. In addition, the terms (terminology) used in this specification are terms used to appropriately express preferred embodiments of the present disclosure, and these may vary depending on the intention of the user or operator, the customs of the field to which the present disclosure belongs, etc.

Therefore, definitions of these terms should be made based on the content throughout this specification. When it is said that a certain part “includes” a certain component throughout the specification, it means that it may further include other components, not excluding other components unless otherwise specifically stated.

The present disclosure provides a compound represented by the following chemical formula 1 or a pharmaceutical salt thereof:

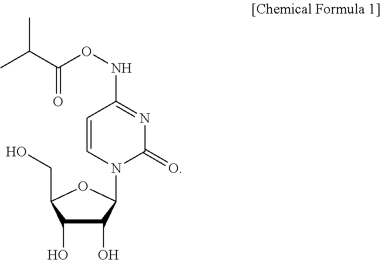

In one embodiment of the present disclosure, the compound may be prepared by a process according to the following reaction scheme, but is not limited thereto:

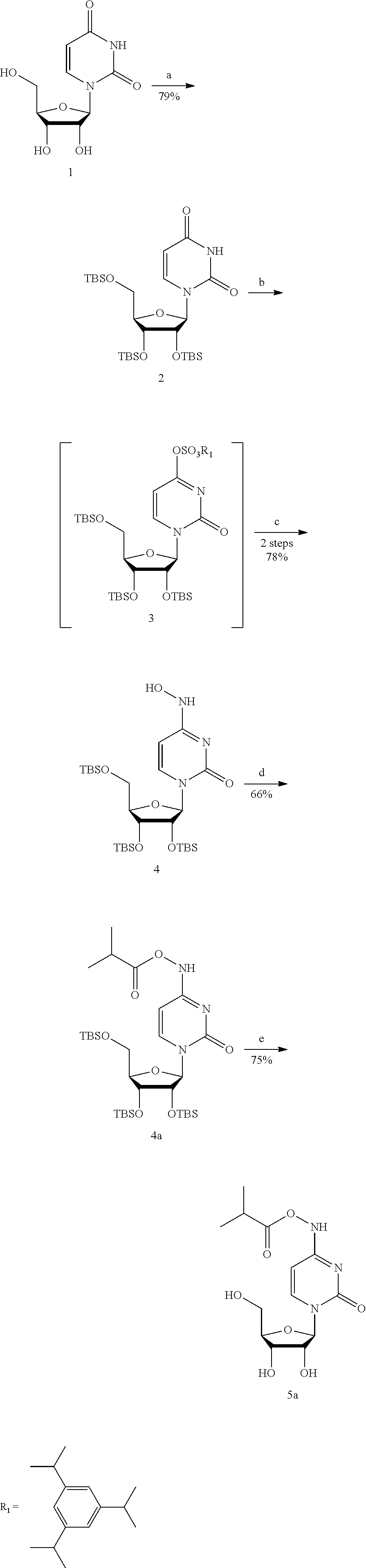

Reagents and reaction conditions: a) TBSCl, imidazole, DMF, 0° C.→rt, 12 h; b) 2,4,6-Triisopropylbenzenesulfonyl chloride, DMAP, DIEA, CH₂Cl₂, 0° C.→rt, 15 h; c) NH₂OH—HCl, DIEA, 0° C.→rt, 12 h; d) Isobutyryl chloride, Et₃N, CH₂Cl₂, 0° C., 12 h; e) 20 equiv. Et₃N-3HF, THF, 0° C.→rt, 48 h.

Further, the present disclosure provides an antiviral composition including the compound according to the present disclosure or a pharmaceutical salt thereof as an active ingredient.

In one embodiment of the present disclosure, the virus may be, but is not limited to, an RNA virus. The virus may be, but is not limited to, severe acute respiratory syndrome coronavirus 2 (SARS-COV-2), dengue virus, or influenza virus. The influenza virus may be, but is not limited to, influenza A or influenza B virus.

In the pharmaceutical composition of the present disclosure, the compound according to the present disclosure may be administered in an appropriate formulation together with carriers and diluents known in the art, and may have a formulation such as oral administration or parenteral administration, for example, intravenous injection, intramuscular injection, intraperitoneal injection, subcutaneous injection, suppository, etc., depending on the intended method.

The above formulations may be prepared by a conventional method using suitable excipients, fillers, binders, wetting agents, disintegrants, lubricants, surfactants, dispersants, buffers, preservatives, solubilizers, disinfectants, sweeteners, flavoring agents, analgesics, stabilizers, isotonic agents, etc., which are conventionally used in pharmaceutical compositions.

Each of the formulations described above may contain pharmaceutically acceptable carriers or additives. Specific examples of the above carriers or additives include water, pharmaceutically acceptable organic solvents, collagen, polyvinyl alcohol, polyvinylpyrrolidine, carboxyvinyl polymers, sodium alginate, water-soluble dextran, sodium carboxymethyl starch, pectin, xanthan gum, gum arabic, casein, gelatin, agar, glycerol, propylene glycol, polyethyl glycol, petrolatum, paraffin, stearyl alcohol, stearic acid, human serum albumin, mannitol, sorbitol, lactic acid, etc. One or more additives may be selected or appropriately combined depending on the formulation. Furthermore, for the cell therapy administration method, local administration to target cells may be performed in addition to conventional systemic administration such as intravenous or intraarterial administration, and an administration method combined with catheter technology and surgical operation may be used.

The composition of the present disclosure may include a compound according to the present disclosure in a pharmaceutically effective amount together with a pharmaceutically acceptable carrier.

In the present disclosure, “pharmaceutically effective amount” refers to the amount of an effective ingredient that exhibits an alleviating, suppressing, improving and/or curing effect on an immune rejection disease to be treated. The range of the dosage of the compound according to the present disclosure varies depending on the patient's weight, age, gender, health condition, diet, administration time, administration method, and severity of disease. For example, a therapeutically effective dose may initially be determined using in vitro assays in cell culture. This field will be able to determine therapeutically effective doses without undue experimentation, and this information may be used to more accurately determine useful doses in humans. For example, the compound according to the present disclosure may be administered as an active ingredient in an amount of 0.1 to 100 mg/kg/day.

The active substance of the present disclosure may be used in the form of a pharmaceutically acceptable salt, and as a salt, an acid addition salt formed by a pharmaceutically acceptable free acid is useful. The expression “pharmaceutically acceptable salt” means any organic or inorganic addition salt of a base compound of an active substance, at a concentration which has an effective effect that is relatively non-toxic and harmless to the patient, and where the side effects attributable to this salt do not diminish the beneficial effects of the base compound of the active substance. These salts may use inorganic acids and organic acids as free acids, and as inorganic acids, hydrochloric acid, bromic acid, nitric acid, sulfuric acid, perchloric acid, phosphoric acid, etc. may be used, and as organic acids, citric acid, acetic acid, lactic acid, maleic acid, fumaric acid, gluconic acid, methanesulfonic acid, glycolic acid, succinic acid, tartaric acid, galacturonic acid, embonic acid, glutamic acid, aspartic acid, oxalic acid, (D) or (L) malic acid, maleic acid, methanesulfonic acid, ethanesulfonic acid, 4-toluenesulfonic acid, salicylic acid, citric acid, benzoic acid, or malonic acid, etc. may be used. Further, these salts include alkali metal salts (sodium salts, potassium salts, etc.), alkaline earth metal salts (calcium salts, magnesium salts, etc.), etc. For example, acid addition salts may include acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methyl sulfate, naphthylate, 2-naphthylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, arginine, benzathine, calcium, choline, diethylamine, diolamine, glycine, lysine, magnesium, meglumine, olamine, potassium, sodium, tromethamine, zinc salts, etc., of which hydrochloride or trifluoroacetate is preferred.

The acid addition salt according to the present disclosure may be prepared by a conventional method, for example, dissolving an effective substance in an organic solvent, such as methanol, ethanol, acetone, methylene chloride, acetonitrile, etc., adding an organic acid or inorganic acid, and filtering and drying the resulting precipitate, or by distilling the solvent and an excess acid under reduced pressure and then drying or crystallizing the same in the presence of an organic solvent.

Further, pharmaceutically acceptable metal salts may be prepared using bases. Alkali metal or alkaline earth metal salts are obtained, for example, by dissolving the compound in an excess of alkali metal hydroxide or alkaline earth metal hydroxide solution, filtering off the undissolved compound salt, and evaporating and drying the filtrate. At this time, it is pharmaceutically suitable to prepare sodium, potassium or calcium salts for the metal salt. Further, the corresponding silver salt is obtained by reacting an alkali metal or alkaline earth metal salt with a suitable silver salt (e.g., silver nitrate).

Furthermore, the present disclosure includes not only the effective substance and its pharmaceutically acceptable salts, but also all possible solvates, hydrates, isomers, optical isomers, etc. that may be prepared therefrom.

In addition, the present disclosure provides a method for preventing or treating a viral infection, which includes administering to a subject a compound according to the present disclosure or a pharmaceutical salt thereof or a composition including the same.

The subject may be a mammal, for example, but is not limited to, a human.

### Modes of the Invention

Hereinafter, the present disclosure is described in detail by examples. However, these examples are intended to explain the present disclosure more specifically, and the scope of the present disclosure is not limited to these examples.

[Preparation Example 1] Preparation of N⁴-isobutyryloxycytidine (5a)

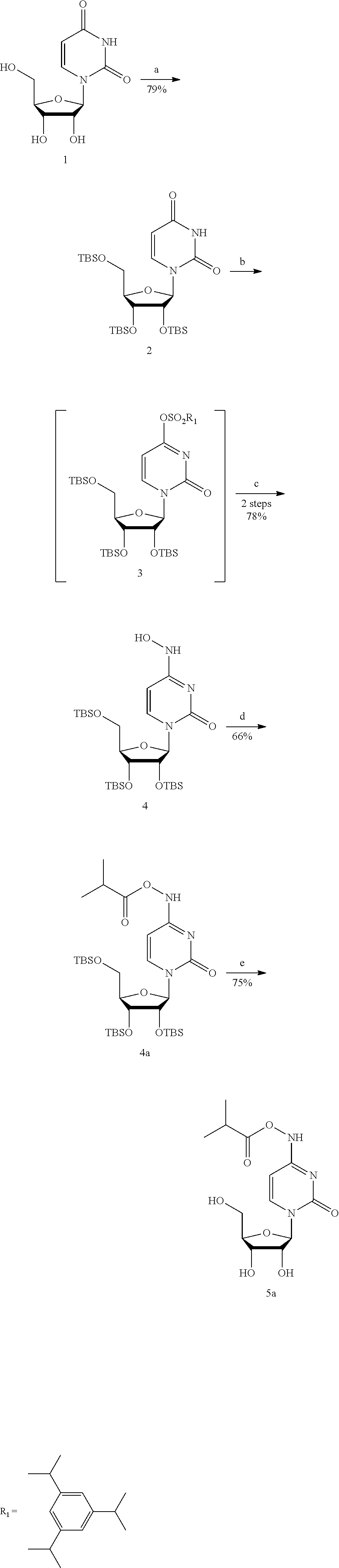

Reagents and reaction conditions: a) TBSCl, imidazole, DMF, 0° C.→rt, 12 h; b) 2,4,6-Triisopropylbenzenesulfonyl chloride, DMAP, DIEA, CH₂Cl₂, 0° C.→rt, 15 h; c) NH₂OH—HCl, DIEA, 0° C.→rt, 12 h; d) Isobutyryl chloride, Et₃N, CH₂Cl₂, 0° C., 12 h; e) 20 equiv. Et₃N-3HF, THF, 0° C.→rt, 48 h

### 1-((2R,3R,4R,5R)-3,4-Bis((tert-butyldimethylsilyl)oxy)-5-(((tert-butyldimethylsilyl)oxy)methyl)tetrahydrofuran-2-yl)pyrimidine-2,4 (1H,3H)-dione (2)

After dissolving uridine (1) (5 g, 20.48 mmol) in 100 mL of anhydrous DMF, TBSCl (11.11 g, 73.71 mmol) and imidazole (6.27 g, 92.14 mmol) were added under a N₂ atmosphere at 0° C. The reaction mixture stirred at room temperature for 12 hours was treated with 10 mL of MeOH and stirred at room temperature for 20 minutes. The solution was then poured into cold water (100 mL) and extracted with diethyl ether (100 mL×3). The combined organic layers were washed with brine (100 mL), dried over Na₂SO₄, and filtered. The filtrate was concentrated in vacuo, and the residue was subjected to silica gel column chromatography (Hexane: EtOAc=50:1 to 5:1 v/v) to obtain Compound 2 (9.52 g, 16.22 mmol) in 79% yield.

¹H NMR (CDCl₃, 400 MHZ) δ 8.36 (br, 1H), 8.02 (d, J=8.4 Hz, 1H), 5.87 (d, J=3.6 Hz, 1H), 5.67 (dd, J=2.4, 8.0 Hz, 1H), 4.13-4.05 (m, 3H), 3.97 (dd, J=1.2, 11.6 Hz, 1H), 3.75 (d, J=11.2 Hz, 1H), 0.93 (s, 9H), 0.89 (s, 9H), 0.88 (s, 9H), 0.13-0.06 (m, 18H).

### 1-((2R,3R,4R,5R)-3,4-Bis((tert-butyldimethylsilyl)oxy)-5-(((tert-butyldimethylsilyl)oxy)methyl)tetrahydrofuran-2-yl)-4-(hydroxyamino)pyrimidin-2 (1H)-one (4)

Compound 2 (1.65 g, 2.18 mmol) was dissolved in 20 mL of anhydrous CH₂Cl₂, and 2,4,6-triisopropylbenzenesulfonyl chloride (1.70 g, 5.62 mmol), DMAP (0.04 g, 0.33 mmol), and N,N-diisopropylethylamine (DIEA) (1.82 g, 14.05 mmol) were added thereto. After stirring at room temperature for 15 hours, the reaction mixture was treated with NH₂OH—HCl (0.78 g, 11.25 mmol) and DIEA (1.45 g, 1.45 mmol) at 0° C. in an N2 atmosphere. After stirring at room temperature for 12 h, the resulting solution was diluted with CH₂Cl₂ (20 mL), and the resultant was immersed in crushed ice (10 g) and stirred for 10 minutes. The resulting solution was poured into a separatory funnel and the organic layer was separated. The aqueous layer was washed with CH₂Cl₂ (20 mL×2). The combined organic layers were washed with brine (100 mL), dried over Na₂SO₄, and filtered. The filtrate was concentrated in vacuo, and the residue was subjected to silica gel column chromatography (Hexane: EtOAc=10:1 to 2:1 v/v) to obtain Compound 4 (1.31 g, 2.18 mmol) in 78% yield.

¹H NMR (CDCl₃, 400 MHZ) δ 8.55 (br, 1H), 7.24 (d, J=8.4 Hz, 1H), 5.90 (d, J=4.4 Hz, 1H), 5.58 (d, J=8.4 Hz, 1H), 4.08-4.02 (m, 2H), 4.00 (d, J=1.6 Hz, 1H), 3.90 (dd, J=11.6, 2.4 Hz, 1H), 3.71 (dd, J=11.56, 1.2 Hz, 1H), 1.79 (s, 1H), 0.93 (s, 9H), 0.90 (s, 9H), 0.88 (s, 9H), 0.11-0.04 (m, 18H): 13C NMR (CDCl₃, 100 MHz) δ 149.49, 145.62, 131.19, 97.95, 87.88, 85.04, 75.40, 71.95, 62.69, 18.58, 18.22, 18.08, −4.20, −4.48, −4.51, −4.63, −5.34, −5.36.

### 1-((2R,3R,4R,5R)-3,4-Bis((tert-butyldimethylsilyl)oxy)-5-(((tert-butyldimethylsilyl)oxy)methyl)tetrahydrofuran-2-yl)-4-((isobutyryloxy)amino)pyrimidin-2 (1H)-one (4a)

Compound 4 (0.52 g, 0.86 mmol) was dissolved in 9 mL of anhydrous CH₂Cl₂, and then isobutyryl chloride (0.12 g, 1.08 mmol) and triethylamine (0.13 g, 1.30 mmol) were added thereto at 0° C. C under an argon atmosphere. After stirring at room temperature for 2 hours, the reaction mixture was treated with 0.2 mL of MeOH to remove excess isobutyryl chloride. After stirring at 0° C. for 5 minutes and concentrating under reduced pressure, the residue was diluted with CH₂Cl₂ (10 mL), and the resultant was adsorbed onto silica gel, and Compound 4a (0.22 g, 0.29 mmol) was obtained in 66% yield by silica gel chromatography (Hexane: EtOAc=8:1 to 5:1 v/v).

¹H NMR (CDCl₃, 400 MHZ) δ 8.28 (br, 1H), 7.51 (d, J=8.0 Hz, 1H), 5.87 (d, J=3.6 Hz, 1H), 5.76 (dd, J=1.6, 8.4 Hz, 1H), 4.07-4.02 (m, 3H), 3.91 (dd, J=2.0, 11.6 Hz, 1H), 3.71 (d, J=11.4 Hz, 1H), 2.72-2.65 (m, 1H), 1.25 (d, J=4.0 Hz, 3H), 1.24 (d, J=3.6 Hz 3H), 0.92-0.86 (m, 27H), 0.10-0.03 (m, 18H): ¹³C NMR (CD₃OD, 100 MHz) δ 173.52, 148.87, 148.68, 133.72, 97.24, 88.22, 85.21, 76.08, 75.76, 71.76, 62.49, 33.02, 26.04, 25.93, 25.83, 19.31, 18.51, 18.20, 18.03, −4.21, −4.56, −4.73, −5.40, −5.54.

### 1-((2R,3R,4S,5R)-3,4-Dihydroxy-5-(hydroxymethyl)tetrahydrofuran-2-yl)-4-((isobutyryloxy)amino)pyrimidin-2 (1H)-one (5a) (N⁴-isobutyryloxycytidine)

Compound 4a (0.10 g, 0.15 mmol) was added to 2 mL of THE, and Et₃N-3HF (0.12 g, 0.74 mmol) was added thereto at 0° C. under an argon atmosphere. After stirring at room temperature for 48 hours, the reaction mixture was carefully treated with silica gel (3.0 g) at 0° C., and the resulting product was stirred at the same temperature for 30 minutes and then concentrated under reduced pressure. The residue was purified by silica gel chromatography (CH₂Cl₂:MeOH=20:1 to 8:1 v/v) to obtain Compound 5a (0.04 g, 0.11 mmol) in 75% yield.

¹H NMR (CD₃OD, 400 MHZ) δ 7.51 (d, J=8.4 Hz, 1H), 5.89 (d, J=5.6 Hz, 1H), 5.73 (d, J=8.0 Hz, 1H), 4.19-4.13 (m, 2H), 3.97 (q, J=3.6 Hz, 1H), 3.81 (dd, J=2.8, 12.0 Hz, 1H), 3.72 (dd, J=3.2, 12.0 Hz, 1H), 2.86-2.83 (m, 1H), 1.23 (d, J=6.8 Hz, 6H); ¹³C NMR (CD₃OD, 100 MHz) δ 176.33, 151.17, 150.84, 135.95, 97.35, 89.94, 86.29, 75.07, 71.62, 64.30, 62.58, 33.55, 19.49; HRMS-ESI⁺: m/z calcd for C₁₃H₁₉N₃O₇ (M+H⁺) 330.1296, found 330.1292.

### Preparation Example 2

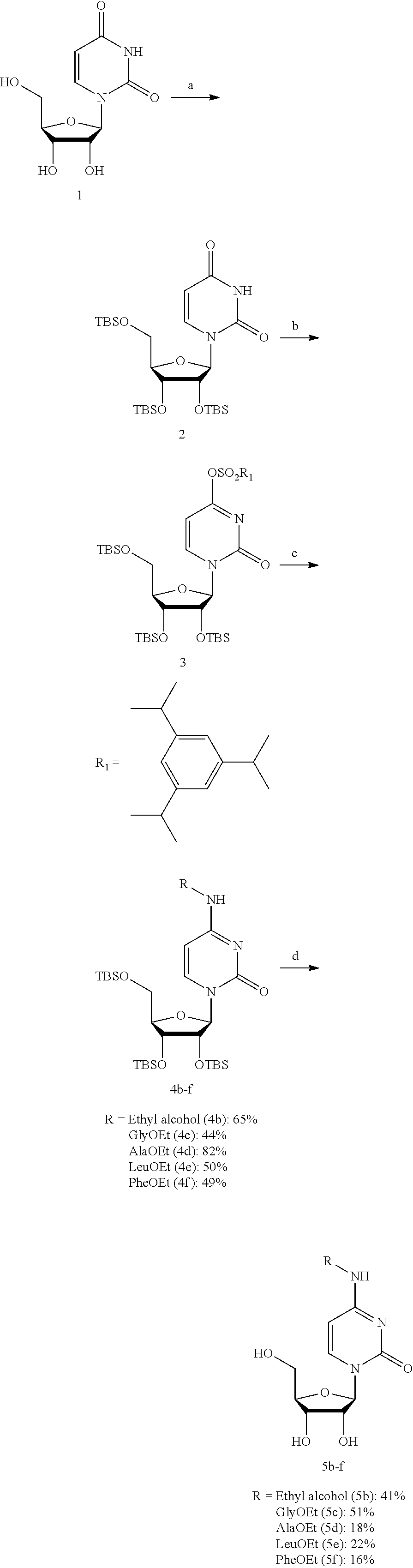

Reagents and reaction conditions: a) TBSCl, imidazole, DMF, 0° C.→rt, 12 h; b) 2,4,6-Triisopropylbenzenesulfonyl chloride, DMAP, DIEA, CH₂Cl₂, 0° C.→rt, 15 h; c) 2-Aminoethanol for 4b, ethyl amino acids-HCl for 4c-f, Et₃N, 0° C.→rt, CH₃CN, 12 h; d) HCl/EtOH, EtOH, rt, 12 h for 5b-f 1-((2R,3R,4R,5R)-3,4-Bis((tert-butyldimethylsilyl)oxy)-5-(((tert-butyldimethylsilyl)oxy)methyl)tetrahydrofuran-2-yl)-4-((2-hydroxyethyl)amino)pyrimidin-2 (1H)-one (4b)

The compound was obtained in 65% yield using a reaction method similar to that for obtaining Compound 4 in Preparation Example 1.

¹H NMR (CDCl₃, 400 MHz) δ 9.30 (br, 0.5H), 8.21 (d, J=7.6 Hz, 0.5H), 8.01 (d, J=7.6 Hz, 0.5H), 6.65 (br, 0.5H), 5.75-5.62 (m, 2H), 4.13-4.00 (m, 5H), 3.86-3.75 (m, 3H), 3.63-3.38 (m, 2H), 0.95-0.87 (m, 27H), 0.13-0.06 (m, 18H).

### 1-((2R,3R,4S,5R)-3,4-Dihydroxy-5-(hydroxymethyl)tetrahydrofuran-2-yl)-4-((2-hydroxyethyl)amino)pyrimidin-2 (1H)-one (5b)

The TBS protecting group was removed from Compound 4b using HCl/EtOH solution to obtain Compound 5b in 41% yield.

¹H NMR (CD₃OD, 400 MHZ) δ 7.93 (d, J=8.0 Hz, 1H), 5.89 (d, J=1.2 Hz, 1H), 5.87 (d, J=1.2 Hz, 1H), 4.15-4.11 (m, 2H), 4.01-4.00 (m, 1H), 3.87 (dd, J=2.8, 12.4 Hz, 1H), 3.74 (dd, J=3.2, 12.4 Hz, 1H), 3.67 (t, J=5.6 Hz, 2H), 3.49 (t, J=5.6 Hz, 2H). ¹³C NMR (CD₃OD, 100 MHz) δ 165.78, 158.81, 141.60, 97.01, 92.26, 85.76, 76.10, 70.79, 62.01, 61.45, 44.17; HRMS-ESI⁺: m/z calcd for C₁₁H₁₇N₃O₆ (M+H⁺) 288.1190, found 288.1188.

### Ethyl(1-((2R,3R,4R,5R)-3,4-bis((tert-butyldimethylsilyl)oxy)-5-(((tert-butyldimethylsilyl)oxy)methyl)tetrahydrofuran-2-yl)-2-oxo-1,2-dihydropyrimidin-4-yl)glycinate (4c)

The compound was obtained in 44% yield using a reaction method similar to that for obtaining Compound 4 in Preparation Example 1.

¹H NMR (CDCl₃, 400 MHZ) δ 8.16 (d, J=7.2 Hz, 1H), 5.71 (s, 1H), 5.60 (d, J=7.2 Hz, 1H), 5.29 (br, 1H), 4.27-4.19 (m, 4H), 4.13-4.00 (m, 4H), 3.76 (d, J=10.8 Hz, 1H), 1.28 (t, J=7.2 Hz, 3H), 0.94 (s, 9H), 0.90 (s, 9H), 0.87 (s, 9H), 0.24-0.03 (m, 18H): ¹³C NMR (CDCl₃, 100 MHz) δ 163.40, 141.12, 93.81, 90.87, 82.71, 69.04. 61.78, 60.79, 42.60, 26.22, 26.06, 26.00, 18.67, 18.20, 14.27, −3.92, −4.01, −4.94, −5.05, −5.14, −5.44.

### Ethyl(1-((2R,3R,4S,5R)-3,4-dihydroxy-5-(hydroxymethyl)tetrahydrofuran-2-yl)-2-oxo-1,2-dihydropyrimidin-4-yl)glycinate (5c)

The compound was obtained in 51% yield using the same method as for obtaining Compound 5b.

¹H NMR (CD₃OD, 400 MHZ) δ 8.70 (d, J=8.0 Hz, 0.2H), 8.47 (d, J=8.0 Hz, 0.8H), 6.32 (d, J=8.0 Hz, 0.2H), 6.22 (d, J=8.0 Hz, 0.8H), 5.88 (m, 1H), 4.34-4.25 (m, 3H), 4.22-4.15 (m, 2H), 4.09-4.06 (m, 1H), 3.92 (m, 1H), 3.77 (m, 1H), 3.60 (q, J=6.8 Hz, 0.8H), 3.48 (q, J=6.8 Hz, 0.2H), 1.33-1.29 (m, 3H): ¹³C NMR (CD₃OD, 100 MHz) δ 168.57, 159.81, 145.10, 99.56, 95.21, 91.95, 86.47, 76.37, 70.52, 63.31, 61.45, 44.79, 14.40; HRMS-ESI⁺: m/z calcd for C₁₃H₁₉N₃O₇ (M+H⁺) 330.1296, found 330.1294.

### Ethyl(1-((2R,3R,4R,5R)-3,4-bis((tert-butyldimethylsilyl)oxy)-5-(((tert-butyldimethylsilyl)oxy)methyl)tetrahydrofuran-2-yl)-2-oxo-1,2-dihydropyrimidin-4-yl)-L-alaninate (4d)

The compound was obtained in 82% yield using a reaction method similar to that for obtaining Compound 4 in Preparation Example 1.

¹H NMR (CDCl₃, 400 MHZ) δ 8.16 (d, J=7.2 Hz, 1H), 5.70 (s, 1H), 5.58 (br, 1H), 5.54 (d, J=7.2 Hz, 1H), 4.97 (t, J=6.8 Hz, 1H), 4.20 (q, J=6.8 Hz, 2H), 4.16-4.06 (m, 3H), 4.11-3.99 (dd, J=8.0, 4.0 Hz, 1H), 3.75 (dd, J=1.2, 12.0 Hz, 1H), 1.45 (d, J=7.2 Hz, 3H), 1.30-1.09 (m, 3H), 0.94-0.87 (m, 27H), 0.25-0.03 (m, 18H).

### Ethyl(1-((2R,3R,4S,5R)-3,4-dihydroxy-5-(hydroxymethyl)tetrahydrofuran-2-yl)-2-oxo-1,2-dihydropyrimidin-4-yl)-L-alaninate (5d)

The compound was obtained in 18% yield using the same method as for obtaining Compound 5b.

¹H NMR (CD₃OD, 400 MHZ) § 8.45 (d, J=8.0 Hz, 1H), 6.19 (d, J=8.0 Hz, 1H), 5.89 (d, J=3.2 Hz, 1H), 4.68 (q, J=7.2 Hz, 1H), 4.32-4.24 (m, 2H), 4.23-4.16 (m, 2H), 4.11-4.07 (m, 1H), 3.91 (dd, J=2.4, 12.4 Hz, 1H), 3.78 (dd, J=2.0, 12.4 Hz, 1H), 1.58 (d, J=7.2 Hz, 3H), 1.31 (t, J=7.2 Hz, 3H).

### Ethyl1-((2R,3R,4R,5R)-3,4-bis((tert-butyldimethylsilyl)oxy)-5-(((tert-butyldimethylsilyl)oxy)methyl)tetrahydrofuran-2-yl)-2-oxo-1,2-dihydropyrimidin-4-yl)-L-leucinate (4e)

The compound was obtained in 50% yield using the similar method as for obtaining Compound 4.

¹H NMR (CDCl₃, 400 MHz) δ 8.16 (d, J=7.6 Hz, 1H), 5.68 (s, 1H), 5.55 (d, J=7.6 Hz, 1H), 5.29 (d, J=8.4 Hz, 1H), 5.13-5.07 (m, 1H), 4.02-4.15 (m, 2H), 4.11-4.06 (m, 3H), 4.02-3.99 (m, 1H), 3.76 (d, J=10.8 Hz, 1H), 1.78-1.72 (m, 1H), 1.69-1.63 (m, 1H), 1.60-1.54 (m, 1H), 1.27 (t, J=3.2 Hz, 3H), 0.94-0.86 (m, 33H), 0.26-0.03 (m, 18H): ¹³C NMR (CDCl₃, 100 MHz) δ 173.83, 163.40, 155.99, 141.06, 93.88, 91.00, 82.49, 76.29, 68.79, 61.53, 60.64, 51.43, 41.88, 29.81, 26.24, 26.06, 26.00, 25.02, 22.99, 22.47, 18.49, 18.19, 14.26, −3.88, −3.94, −4.96, −5.07, −5.14, −5.45.

### Ethyl(1-((2R,3R,4S,5R)-3,4-dihydroxy-5-(hydroxymethyl)tetrahydrofuran-2-yl)-2-oxo-1,2-dihydropyrimidin-4-yl)-L-leucinate (5e)

The compound was obtained in 22% yield using the same method as for obtaining Compound 5b.

¹H NMR (CD₃OD, 400 MHZ) δ 8.72 (d, J=8.0 Hz, 0.2H), 8.45 (d, J=8.0 Hz, 0.8H), 6.38 (d, J=8.0 Hz, 0.2H), 6.17 (d, J=8.0 Hz, 0.8H), 5.88 (d, J=3.2 Hz, 0.8H), 5.87 (d, J=2.8 Hz, 0.2H), 4.68 (dd, J=4.4, 9.6 Hz, 0.8H), 4.62 (dd, J=8.4, 5.6 Hz, 0.2H), 4.29-4.22 (m, 2H), 4.22-4.15 (m, 2H), 4.10-4.06 (m, 1H), 3.91 (dd, J=12.4, 2.4 Hz, 1H), 3.77 (dd, J=12.4, 2.8 Hz, 1H), 1.91-1.69 (m, 3H), 1.30 (t, J=7.2 Hz, 3H), 1.01 (d, J=6.4 Hz, 3H), 0.97 (d, J=6.8 Hz, 1H); ¹³C NMR (CD₃OD, 100 MHz) δ 171.25, 157.86, 144.98, 98.56, 95.28, 92.01, 86.42, 76.28, 70.54, 63.40, 61.49, 55.06, 41.00, 26.08, 23.17, 21.53, 14.40; HRMS-ESI⁺: m/z calcd for C₁₇H₂₇N₃O₇ (M+H⁺) 386.1922, found 386.1918.

### Ethyl(1-((2R,3R,4R,5R)-3,4-bis((tert-butyldimethylsilyl)oxy)-5-(((tert-butyldimethylsilyl)oxy)methyl)tetrahydrofuran-2-yl)-2-oxo-1,2-dihydropyrimidin-4-yl)-L-phenylalaninate (4f)

The compound was obtained in 49% yield using a method similar to that for obtaining Compound 4 in Preparation Example 1.

¹H NMR (CDCl₃, 400 MHZ) δ 8.18 (d, J=7.6 Hz, 1H), 7.27-7.22 (m, 3H), 7.05-7.04 (m, 2H), 5.72 (s, 1H), 5.53 (d, J=7.6 Hz, 1H), 5.40 (d, J=8.0 Hz, 1H), 5.32-5.28 (m, 1H), 4.22-4.14 (m, 2H), 4.13-4.08 (m, 3H), 4.01 (q, J=4.0 Hz, 1H), 3.77 (d, J=10.4 Hz, 1H), 3.35 (dd, J=5.6, 10.0 Hz, 1H), 3.17 (dd, J=4.0, 13.6 Hz, 1H), 1.26 (t, J=6.4 Hz, 3H), 0.93-0.87 (m, 27H), 0.28-0.03 (m, 18H): ¹³C NMR (CDCl₃, 100 MHz) δ 171.99, 162.84, 156.06, 141.23, 136.05, 129.66, 128.54, 127.08, 93.91, 91.03, 82.66, 76.34, 68.92, 61.76, 60.71, 53.75, 37.49, 26.24, 26.14, 26.08, 26.01, 25.95, 25.86, 18.67, 18.22, 18.20, 14.28, −3.89, −3.94, −4.96, −5.06, −5.14, −5.43.

### Ethyl(1-((2R,3R,4S,5R)-3,4-dihydroxy-5-(hydroxymethyl)tetrahydrofuran-2-yl)-2-oxo-1,2-dihydropyrimidin-4-yl)-L-phenylalaninate (5f)

The compound was obtained in 18% yield using the same method as for obtaining Compound 5b.

¹H NMR (CD₃OD, 400 MHZ) δ 8.60 (d, J=8.0 Hz, 0.3 H), 8.37 (d, J=7.6 Hz, 0.7 H), 7.33-7.23 (m, 5H), 6.18-6.15 (m, 1H), 5.82-5.80 (m, 1H), 5.02-4.96 (m, 0.7H), 4.94-4.88 (m, 0.3H), 4.13-4.24 (m, 2H), 4.17-4.12 (m, 2H), 4.09-4.04 (m, 1H), 3.92 (d, J=12.4 Hz, 1H), 3.76 (d, J=12.4 Hz, 1H), 3.49-3.36 (m, 1H), 3.19-3.16 (m, 0.3H), 3.14-3.06 (m, 0.7H), 1.17 (t, J=7.2 Hz, 2.1H), 1.24 (t, J=7.2 Hz, 0.9H); ¹³C NMR (CD₃OD, 100 MHz) δ 170.09, 145.06, 136.75, 130.78, 130.38, 129.99, 129.87, 128.55, 95.11, 92.03, 86.39, 76.24, 70.46, 70.08, 63.53, 62.37, 61.44, 57.86, 38.59, 14.40; HRMS-ESI⁺: m/z calcd for C₂₀H₂₅N₃O₇ (M+H⁺) 420.1765, found 420.1762.

### [Example 1] DENV-2 Activity Evaluation Method

### DENV-2 Replicon BHK-21 Cell

Replicon cell lines derived from baby hamster kidney (BHK-21) cells were cultured in Dulbecco's modified eagle's medium (DMEM, Corning) supplemented with 10% fetal bovine serum (FBS, Hyclone) and 5 μg/ml of puromycin (Invivogen) at 37° C. and 5% or less CO₂. Antiviral and cytotoxic effects were verified using culture medium without antibiotics.

### Cytotoxicity Assay

The cytotoxicity of the compound prepared in Preparation Example 1 was evaluated in DENV-2 replicon BHK-21 (DENV2-BHK) cells using the MTT (3-(4,5-dimethylthiazol-2-yl)-2,5-diphenyltetrazolium bromide, Invitrogen, M6494) assay. In this study, BHK-21 cells were prepared at 5,000 cells/well in a 96-well plate at 37° C. under 5% CO₂. After the cells were attached, three test compounds were injected at the same concentration (10 μM), it was cultured, and then the resultant was treated with MTT solution (0.5 mg/ml) and cultured for 30 minutes. After removing the supernatant, DMSO was added thereto to dissolve the indicator as formazan (purple), and its absorbance value was measured at 540 nm using a spectrophotometer (Spectrophotometer Plus 384, Molecular Devices Corp, CA, Sunnyvale, Molecular Devices Corp). Results are expressed as the mean±S.E. of three repeated tests.

### Luciferase Reporter Assay

Luminescence signals were measured using the Renilla luciferase assay system (Promega). DENV2-BHK was plated at 5,000 cells/well in a 96-well plate with a clear, flat bottom (Corning). The process for processing the compound was as described above, and 40 μl of the supernatant was removed after reaching 90 to 95% confluence. Renilla luciferase assay reagent was added thereto, and the luminescence signal (LS) was measured after 1 to 2 minutes, indicating the maximum value of LS. Final luminescence values were calculated compared to the LS of the untreated group. Results are expressed as the mean±S.E. of three repeated tests.

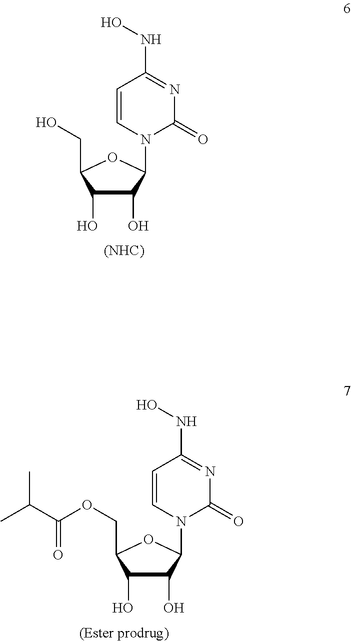

An ester prodrug (5a) of Compound 6 (NHC, β-D-N⁴-hydroxycytidine) was evaluated against DENV-2 replicon in vitro. The results of anti-DENV-2 activity and cytotoxicity are shown in Table 2. When the synthetic prodrug (5a) was tested as a replicon, its cytotoxicity was reduced by 2-fold compared to the parent Compound 6, and its EC₅₀ value was slightly increased by about 2-fold, as shown in Table 1. Additionally, Compound 5a showed an improved EC₅₀ value about 6-fold over the ester precursor 7. However, other synthetic compounds, including ethyl alcohol derivatives (5b) and C₄-amino acid derivatives (5c-f), did not exhibit significant anti-DENV activity in replication assays.

TABLE 1
Anti-DENV-2 activity and cytotoxicity
of nucleoside analogues in BHK-21 cells

| Entry | Compound | EC₅₀(μM) | CC₅₀(μM) | S.I. |
|---|---|---|---|---|
| 1 | RBV | 1.16 | 5.46 | 4.70 |
| 2 | 6 | 2.84 | 5.75 | 2.02 |
| 3 | 5b | >100 | >100 | — |
| 4 | 5c | >100 | >100 | — |
| 5 | 5d | >100 | >100 | — |
| 6 | 5e | >100 | >100 | — |
| 7 | 5f | >100 | >100 | — |
| 8 | 5a | 3.95 | 10.85 | 2.74 |
| 9 | 7 | 23.38 | 42.25 | 1.81 |

RBV, Ribavirin.
EC₅₀, 50% effective concentration.
CC₅₀, 50% cytotoxic concentration.
S.I., Selectivity index = CC₅₀/EC₅₀.

### [Example 2] SARS-COV-2 Activity Evaluation

### Cell, Virus and Compound

SARS-COV-2 (BetaCoV/Korea/KCDC03/2020) provided by the Korea Disease Control and Prevention Agency was amplified in Vero cells at 37° C. for 3 days. After centrifugation at 1000 g for 5 minutes, the virus was stored at −80° C., and the virus titer was measured by plaque assay.

### Cell Culture-Based Antiviral Assay

To assess anti-SARS-COV-2 activity in an image-based system, Vero cells were cultured overnight in 96-well plates (2×10⁴ cells per well). After diluting the compound 3-fold, cells were infected with the same amount of SARS-COV-2 (MOI 0.05) at 37° C. for 2 days in a biosafety level 3 laboratory. Cells were fixed with refrigerated acetone:methanol (1:3), permeabilized, probed with anti-spike antibody (Genetex, Irvine, CA, USA), and EC₅₀ values were determined with Alexa Fluor 488-conjugated goat anti-mouse IgG (Invitrogen, Carlsbad, CA, USA). Cell nuclei were counterstained with 4′,6-diamidino-2-phenylindole (DAPI: Invitrogen) to calculate CC50 values. The number of viral spike protein-induced or cell nucleus-derived signals detected at four points per well was quantified in three independent samples using the Operetta High Content Screening System (Perkin Elmer, Waltham, MA, USA) and the embedded Harmony High Content Imaging and Analysis Software 3.5.2. To determine the 50% tissue culture infectious dose (TCID₅₀), cells infected with SARS-COV-2 were cultured for 2 days in the absence or presence of antiviral compounds. Fresh Vero cells seeded in 96-well plates were infected with serial 10-fold dilutions of culture supernatant for 2 days. TCID₅₀ was determined by counting the number of SARS-CoV-2 spike protein-induced green fluorescent populations and DAPI-induced nuclear distributions as described above.

### Biological Activity Against SARS-COV-2

The ester prodrug derivative (5a) was investigated against SARS-COV-2 in vitro. The results of activity and cytotoxicity of SARS-COV-2 are summarized in Table 2. When the synthetic precursor (5a) was tested with SARS-COV-2 (BetaCoV/Korea/KCDC03/2020), the antiviral activity of the ester prodrug (5a) was improved by about 5 times and the cytotoxicity was improved by about 6 times or more compared to the parent Compound 6. The EC₅₀ (effective concentration 50%) value of the new prodrug, 5a, was 3.5 μM, and cytotoxicity was observed at over 100 μM. In particular, the antiviral activity of Compound 5a was improved 3.5-fold over that of the other ester precursor 7, and the cytotoxicity values were similar to each other. However, other synthetic compounds, including ethyl alcohol derivatives (5b) and C4-amino acid derivatives (5c-f), did not show significant biological SARS-COV-2 activity.

TABLE 2
Anti-SARS-CoV-2 physiological activity and cytotoxicity

| Entry | Compound | EC₅₀(μM) | CC₅₀(μM) | SI |
|---|---|---|---|---|
| 1 | 6 | >16.5 | 16.5 | — |
| 2 | 5b | >100 | >100 | — |
| 3 | 5c | >100 | >100 | — |
| 4 | 5d | >100 | >100 | — |
| 5 | 5e | >100 | >100 | — |
| 6 | 5f | >100 | >100 | — |
| 7 | 5a | 3.5 | >100 | >28.6 |
| 8 | 7 | 11.8 | >100 | >8.5. |
| 9 | Remdesivir | 11.1 | >100 | >9.0 |

### [Example 3] Anti-Influenza Activity Evaluation

### Cell, Virus and Compound

Influenza viruses A/Puerto Rico/8/34 (PR8; HIN1), A/Hong Kong/8/68 (HK: H3N2), and B/Lee/40 (Lee) were purchased from ATCC. Influenza A virus was inoculated into 10-day-old embryonated oocytes at 37° C. for 3 days, whereas influenza B virus was amplified in MDCK cells in the presence of 2 μg/ml of phenylalanyl chloromethyl ketone (TPCK)-treated trypsin (Sigma-Aldrich, St. Louis, MO, USA) at 35° C. for 3 days.

### Cell Culture-Based Antiviral Assay

Antiviral assays against influenza virus were performed according to a protocol in which MDCK cells grown overnight in 96-well plates (3×10⁴ cells per well) were mock-infected or infected with each virus strain at a multiplicity of infection (MOI) of 0.001 for 1 hour at 35° C. For the viability of Illations. non-infected or infected cells for each compound at the same temperature for 3 days, half maximal cytotoxic concentration (CC₅₀) and half maximal effective concentration (EC₅₀), respectively, were measured using 3-(4,5-dimethylthiazol-2-yl)-2,5-diphenyltetrazolium bromide.

### Biological Activity Against Influenza A and B Viruses

As summarized in Table 3, the novel ester prodrug (5a) was evaluated against Flu A/Puerto Rico/8/34 (PR8; HIN1), Flu A/Hong Kong/8/68 (HK: H3N2), and Flu B/Lee/40 (Lee). Efficacy results and cytotoxicity for antitoxic A/B are summarized in Table 3. Synthetic Precursor 5a exhibited the same cytotoxicity as Compound 6 and Precursor 7. Additionally, the physiological activity of synthetic Precursor 5a against Flu A (H1N1 and H3N2) was lower than that of parent Compound 6, but showed a similar efficacy value to that of Precursor Compound 7. Conversely, the physiological activity of synthetic Precursor 5a against Flu B was similar to that of parent Compound 6, but its efficacy value was lower than that of Precursor Compound 7. The efficacy of the new ester derivative 5a was 5.8 UM against Flu A (H1N1), 7.3 μM against Flu A (H3N2), and 3.4 μM against Flu B, which values showed a similar trend in anti-influenza efficacy to T T-705 (6-fluoro-3-hydroxy-2-pyrazinecarboxamide). Additionally, the efficacy of derivative 5a showed similar efficacy to that of AMT (amantadine), which is currently used as a treatment for Flu B. However, other synthetic compounds, including ethyl alcohol (5b) and C4-amino acid derivatives (5c-f), did not exhibit significant anti-Flu A/B activity.

TABLE 3
Anti-Flu A and B physiological activity and cytotoxicity

| | | | Antiviral activity (EC₅₀ μM) | | | Selectivity indext | | |
|---|---|---|---|---|---|---|---|---|
| | | Toxicity | Flu A | Flu A | Flu B | Flu A | Flu A | Flu B |
| | | (CC₅₀ | H1N1 | H3N2 | — | H1N1 | H3H2 | — |
| No. | Code | μM) | PR8 | Hong Kong | Lee | PR8 | Hong Kong | Lee |
| 1 | 6 | >100.0 | 1.7 | 2.3 | <1.2 | >58.8 | >43.5 | <1,200 |
| 2 | 5a | >100.0 | 5.8 | 7.3 | 3.4 | >17.2 | >13.7 | >29.4 |
| 3 | 7 | >100.0 | 6.6 | 6.0 | 23.8 | >15.2 | >16.7 | >4.2 |
| 4 | 5b | >100.0 | >100.0 | >100.0 | >100.0 | — | — | — |
| 5 | 5c | >100.0 | >100.0 | >100.0 | >100.0 | — | — | — |
| 6 | 5d | >100.0 | >100.0 | >100.0 | >100.0 | — | — | — |
| 7 | 5e | >100.0 | >100.0 | >100.0 | >100.0 | — | — | — |
| 8 | 5f | >100.0 | >100.0 | >100.0 | >100.0 | — | — | — |
| 9 | AMT | >100.0 | >100.0 | 6.4 | >100.0 | ND | >15.6 | ND |
| 10 | RBV | >100.0 | 37.7 | 38.7 | 33.3 | >2.7 | >2.6 | >3 |
| 11 | T-705 | >100.0 | 3.2 | 8.5 | 7.6 | >1,111 | >20,000 | >13.2 |

AMT: Amantadine
RBV: Ribavirin
T-705: Favipiravir (6-fluoro-3-hydroxy-2-pyrazinecarboxamide)

So far, the present disclosure has been described with a focus on preferred embodiments thereof. A person having ordinary skill in the art to which the present disclosure pertains will understand that the present disclosure can be implemented in modified forms without departing from the essential characteristics of the present disclosure. Therefore, the disclosed examples should be considered from an illustrative rather than a restrictive perspective. The scope of the present disclosure is indicated not by the above description but by the claims, and all differences within the equivalent scope should be interpreted as being included in the present disclosure.

### INDUSTRIAL APPLICABILITY

The N⁴-isobutyryloxycytidine of the present disclosure has improved physiological activity compared to the parent NHC, exhibits higher cytotoxicity and antiviral efficacy against viruses, and thus can be used as an effective antiviral agent.

1. A compound represented by the following Chemical Formula 1 or a pharmaceutical salt thereof:

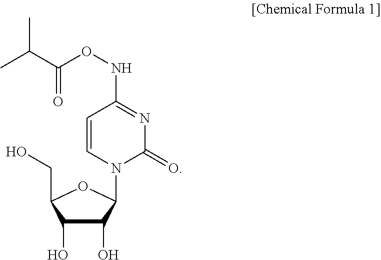

2. A composition for treating viral infection, comprising the compound or the pharmaceutical salt thereof according to claim 1 as an active ingredient.

3. The antiviral composition according to claim 2, wherein the virus is an RNA virus.

4. The antiviral composition according to claim 2, wherein the virus is severe acute respiratory syndrome coronavirus 2 (SARS-COV-2).

5. The antiviral composition according to claim 2, wherein the virus is an influenza virus.

6. The antiviral composition according to claim 5, wherein the influenza virus is an influenza A virus or an influenza B virus.

7. The antiviral composition according to claim 2, wherein the virus is a dengue virus.

8. A method of treating viral infection, the method comprising administering to a subject in need thereof the compound or the pharmaceutical salt thereof according to claim 1 as an active ingredient.
